(19) United States
(12) Patent Application Publication
(10) Pub. No.: US 2022/0346975 A1
(43) Pub. Date: Nov. 3, 2022

(54) IMPLANT SIZING INSTRUMENT

(71) Applicant: CARTIVA, INC., Alpharetta, GA (US)

(72) Inventor: Moriah Ellen MATTSON, Alpharetta, GA (US)

(73) Assignee: CARTIVA, INC., Alpharetta, GA (US)

(21) Appl. No.: 17/761,354

(22) PCT Filed: Dec. 14, 2020
(86) PCT No.: PCT/US2020/050610

§ 371 Date: Mar. 17, 2022

Related U.S. Application Data
(60) Provisional application No. 62/953,239, filed on Dec. 24, 2019.

(51) Int. Cl. A61F 2/46

(52) U.S. Cl. CPC A61F 2/4657; A61F 2002/30125

(57) ABSTRACT

A multi-function device useful in a surgical procedure for implanting a cartilage repairing implant in a bone is disclosed. The device includes a plurality of bone defect sizing rings or discs, where each bone defect sizing ring/disc includes a portion that is used to determine the size that correlates to the perimeter shape and size of the cartilage repairing implant. The inventive device also includes a feature that can be used to gauge the proudness of the cartilage repairing implant after the implant has been implanted into a bone.

### FIELD OF DISCLOSURE

The present disclosure relates generally to orthopedic surgical instruments, and more specifically, to instruments for determining the appropriate size of the cavity to be drilled into a bone that will receive a cartilage repairing implant.

### BACKGROUND

In order to install a cartilage surface repairing implant in an articulating surface of a bone in a joint, a cavity is formed into the cartilage surface of the bone in the joint for receiving the implant. The size of the implant is determined by the size of the cartilage repair site on the cartilage surface. Preferably, the cavity is of an appropriate size that correlates to the size of the cartilage repairing implant.

### SUMMARY

An embodiment of a multi-function device configured for use in a surgical procedure for implanting a cartilage repairing implant in a bone is disclosed. In some embodiments, the device comprises a plurality of bone defect sizing rings, where each bone defect sizing ring comprises a loop that defines an opening of a predefined size that correlates to the perimeter shape and size of a cartilage repairing implant. The openings can have different predefined sizes, for determining the appropriate size of the cartilage repairing implant for a cartilage repair site in a bone.

Also disclosed is an embodiment of a multi-function device that comprises a plurality of bone defect sizing discs, where each bone defect sizing disc has diameter of a predefined size that correlates to the diameter of a cartilage repairing implant. The discs have a first major surface and a second major surface. In some embodiments, one of the first and second major surfaces is concave for contacting a cartilage repair site in a bone, and the discs have different predefined diameters, for determining the appropriate size of the cartilage repairing implant for the cartilage repair site.

### BRIEF DESCRIPTION OF THE DRAWINGS

The various embodiments of the inventive subject matter of the present disclosure will be described in more detail in conjunction with the following drawing figures. The structures in the drawing figures are illustrated schematically, and they are not necessarily drawn to scale. The drawings figures are not intended to show actual dimensions.

FIG. 1 is a perspective view of the bottom side of the multi-function device according to an embodiment of the present disclosure shown from the bottom side.

FIG. 2 is a perspective view of the top side of the multi-function device of FIG. 1.

FIG. 3 is a top-down view of the multi-function device of FIG. 1.

FIG. 4 is an elevation view of the multi-function device of FIG. 1.

FIG. 5 is a perspective view of a multi-function device according to another embodiment.

FIG. 6 is an elevation view of the multi-function device of FIG. 5.

FIG. 7 is a perspective view of a multi-function device according to another embodiment.

FIG. 8 is an illustration showing a variation in the shape of a sizing ring on a multi-function device according to another embodiment.

FIG. 9 is a longitudinal cross-section view of a metatarsal bone B1 where its articulating cartilage surface B1′ has been repaired with a cartilage repairing implant 500.

FIG. 10 is a perspective view of a multi-function device according to another embodiment shown from the bottom side.

FIG. 11 is a perspective view of the multi-function device of FIG. 10 shown from the top side.

FIG. 12 is an elevation view of the multi-function device of FIG. 10.

FIG. 13 is a perspective view of a multi-function device according to another embodiment shown from the bottom side.

FIG. 14 is a perspective view of the multi-function device according to another embodiment shown from the top side.

FIG. 15 is a perspective view of the multi-function device according to another embodiment shown from the bottom side.

FIGS. 16-17 are perspective views of the multi-function device according to various other embodiments shown from the top side.

FIG. 18 is an elevation view of the multi-functional devices of FIGS. 16 and 17.

FIG. 19 is an illustration showing the multi-function device of FIG. 1 being used on a metatarsal bone.

### DETAILED DESCRIPTION

This description of the exemplary embodiments is intended to be read in connection with the accompanying drawings, which are to be considered part of the entire written description. The drawing figures are not necessarily to scale, and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

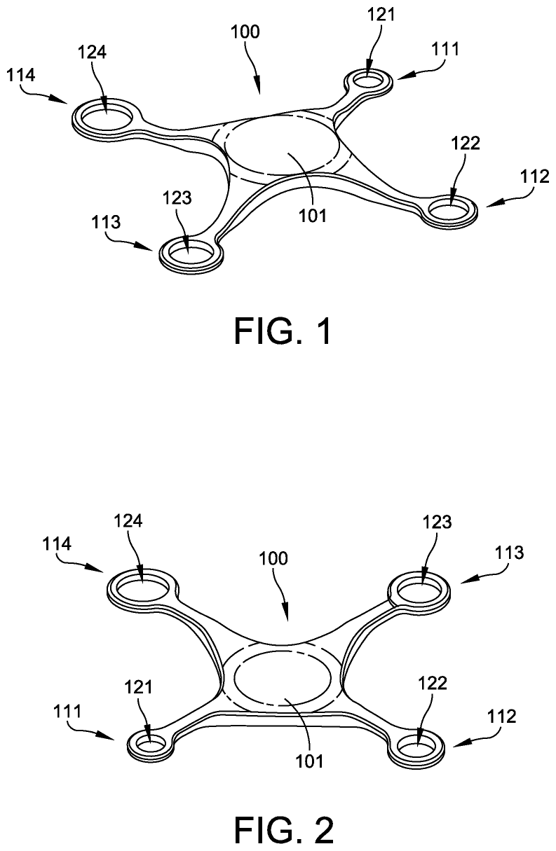

Referring to FIG. 1, according to some embodiments, a multi-function device 100 configured for use in a surgical procedure for implanting a cartilage repairing implant is disclosed. The device 100 comprises a body 101 and a plurality of bone defect sizing rings 111, 112, 113, 114. Each bone defect sizing ring 111, 112, 113, 114 defines an opening 121, 122, 123, 124, respectively, of a predefined size that represents the size of the cavity that needs to be formed in the cartilage surface of the bone to remove a desired amount of the damaged portion of the cartilage and subsequently place a cartilage repairing implant. Thus, the predefined size of the openings 121, 122, 123, 124 of the sizing rings correlates to the perimeter shape and size of the cartilage repairing implant.

In some embodiments, the shape of the opening 121, 122, 123, 124 is circular as in the illustrated example and the diameter of the opening is such that it represents the diameter of a drill bit that would be used to drill a cavity into the cartilage of the bone, such as a first metatarsal. Each opening 121, 122, 123, 124 in the multi-function device 100 can have a different size so that the multi-function device 100 provides a selection of multiple drill bit diameters to choose from to match the size of the damaged area in the cartilage.

A surgeon compares the openings 121, 122, 123, 124 in the sizing rings 111, 112, 113, 114 to the damaged area of the cartilage surface and selects the sizing ring whose opening best circumscribes the damaged area of the cartilage. To determine which of the bone defect sizing rings 111, 112, 113, 114 on the device best matches the size of the damaged area of the cartilage surface, a sizing ring is positioned over the cartilage repair site on the bone to see whether the opening in that sizing ring appropriately encompasses the cartilage repair site.

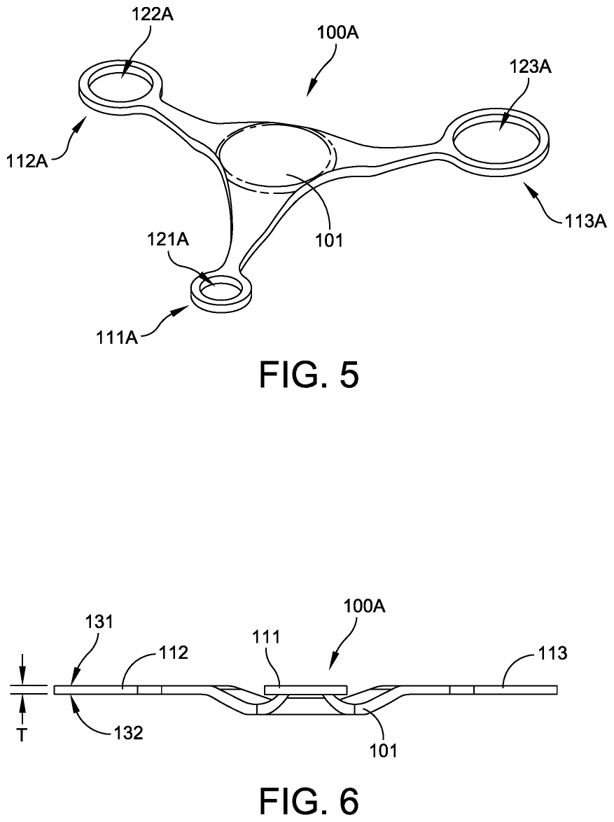

The illustrated example of multi-function device 100 has four bone defect sizing rings 111, 112, 113, and 114. However, in some embodiments, the multi-function device can comprise a different number of bone defect sizing rings. For example, a multi-function device can comprise two to six bone defect sizing rings. In some other embodiments, the multi-function device comprises three to five bone defect sizing rings. For example, FIG. 5 is an illustration of a multi-function device 100A that has three sizing rings, 111A, 112A, and 113A.

Regardless of how many sizing rings are provided on a given multi-function device, each of the sizing rings defines an opening of a predefined size that represents the diameter of a drill bit that would be used to drill a cavity into the cartilage of the bone, such as a first metatarsal. Accordingly, each of the sizing rings on a given multi-function device have different predefined diameters for determining the appropriate size of the cartilage repairing implant for the cartilage repair site.

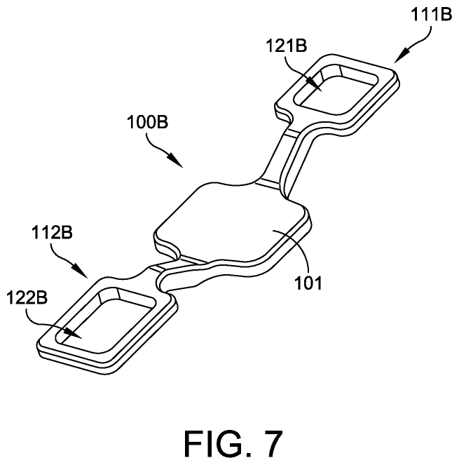

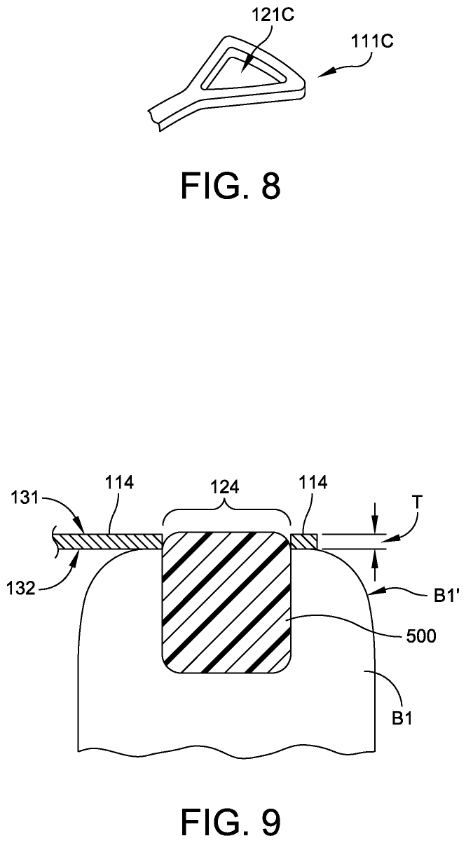

In the embodiments of the multi-function devices 100 and 100A, the bone defect sizing rings and the corresponding openings have a circular shape. In other embodiments, the bone defect sizing rings and the corresponding openings can have a non-circular shape such as an oval, a triangle, or a quadrilateral shape such as a square, a rectangle, a parallelogram, etc. FIGS. 7 and 8 are illustrations of such other shapes. FIG. 7 shows a multi-function device 100B comprising two substantially rectangular shaped bone defect sizing rings 111B and 112B and their correspondingly shaped openings 121B and 122B. FIG. 8 shows an illustration of a bone defect sizing ring 111C and its corresponding opening 121C that have substantially triangular shape. In such embodiments, the size and shape of the openings in the sizing rings would not represent the diameter of a drill bit. One may have to use a smaller drill bit or other suitable bone removing burr to cut a non-circular cavity in the bone. The openings in the sizing rings, however, still correlate to the perimeter shape and size of the cartilage implant that would be implanted into the resulting cavity.

In a preferred embodiment, the multi-function device is made of a transparent or substantially transparent material so that when the sizing rings are placed over the cartilage surface of the bone and are being used to determine the appropriate size that encompasses the damaged cartilage region, one can see through the sizing rings and see the cartilage surface behind the sizing rings. This can be helpful in determining whether the opening in the sizing ring properly encompass the damaged region on the cartilage surface.

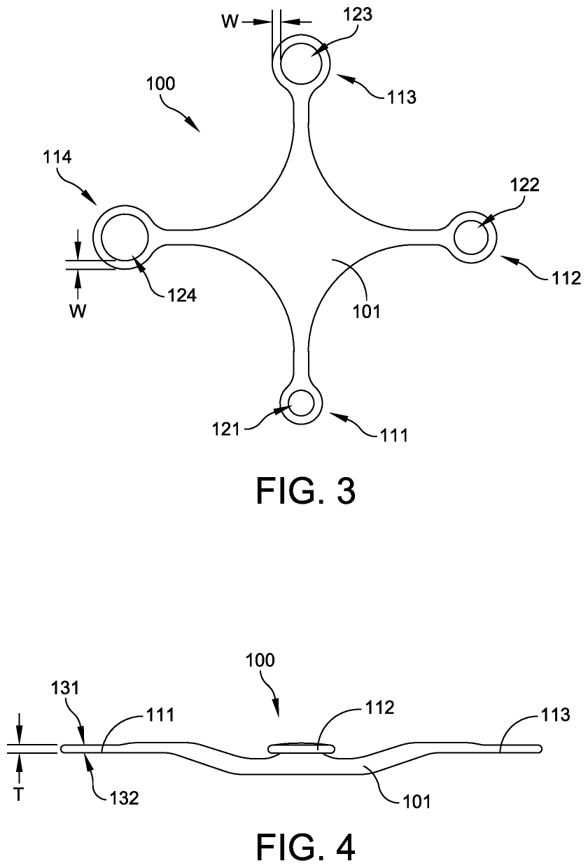

Referring to FIG. 3, in some embodiments of the multi-function device each sizing ring 111, 112, 113, 114 has a predefined width W for assessing the width or amount of the bone material surrounding the intended location of the cartilage repairing implant. As the size of the cavity to be drilled into the cartilage surface of a bone is being determined, it is also useful to be able to determine, how much of the bone material will remain after the cavity for the implant is formed. This is helpful for confirming that there will be sufficient amount of bone material where the implant is intended to be placed. This is particularly useful when the cartilage surface being repaired is at the end of a bone such as a metatarsal. For structural reasons, it is desired that some minimum amount of bone material remains around the cavity being created for the cartilage repairing implant. If not enough bone material remains, the structural integrity of the metatarsal can be compromised and insufficient for viability of the implant operation. For this reason, the widths of the sizing rings are configured to be a predefined width W. In some embodiments, the predefined width W of the sizing rings can range from about 2 mm to 3 mm. In some embodiments, the predefined width of the sizing rings is about 2 mm.

Referring to FIG. 4, another aspect of the multi-function device is that each of the plurality of bone defect sizing rings that define the corresponding openings have a top surface 131 and a bottom surface 132 that define a predefined thickness T between the top and bottom surfaces 131, 132. The thickness T of the sizing ring is used for assessing the proudness of the cartilage repairing implant after the implant has been implanted in the bone.

FIG. 9 shows a longitudinal cross-section view of a metatarsal bone B1 where its articulating cartilage surface B1′ has been repaired with a cartilage repairing implant 500. Generally, the cartilage repairing implant 500 protrudes from the surface B1′ of the cartilage to some degree. In this example, the sizing ring 114 of the multi-function device 100 is being used to measure or gauge the proudness of the implant 500 by comparing the proudness to the thickness T of the sizing ring 114 as the reference dimension. The sizing ring 114 can be placed next to the implant 500 to make the comparison. Alternatively, a sizing ring that is larger than the implant 500 can be positioned over the implant 500 so that the protruding portion of the imiplant 500 sits within the opening of the sizing ring. In the illustrated example, the sizing ring 114 is used to check the proudness. The thickness T of the sizing ring is defined between the top and bottom surfaces 131, 132 of the sizing ring 114. With the protruding portion of the implant 500 positioned within the opening 121, the proudness of the implant can be gauged visually or tactilely against the predefined thickness T of the sizing ring 114. The predefined thickness T of the sizing ring can represent a desired reference point for the proudness of the implant 500 whether it be a maximum proudness, a minimum proudness, or some other target proudness, depending on the need. Therefore, in some embodiments, each sizing ring on the multi-function device can have different predefined thickness. This enables a single multi-function device to be used for gauging different proudness.

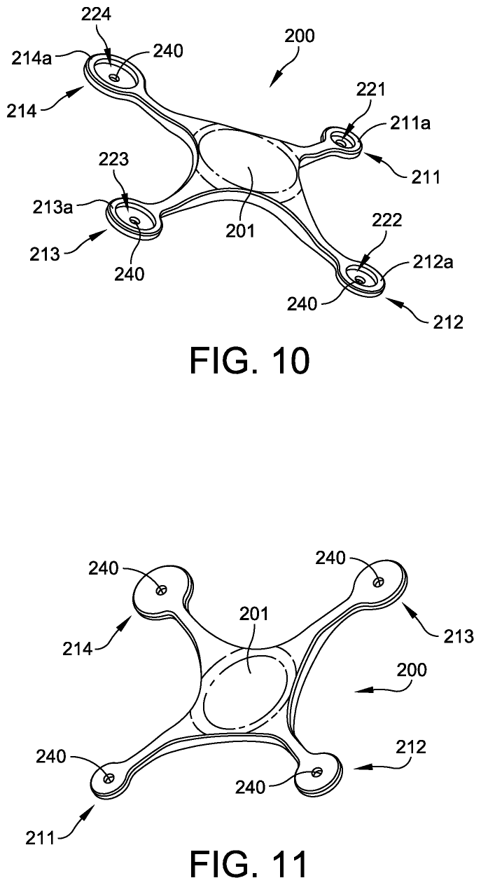

Referring to FIGS. 10-12, a multi-function device 200 according to another embodiment comprises a body 201 and a plurality of bone defect sizing discs 211, 212, 213, 214, where each bone defect sizing disc has diameter of a predefined size that correlates to the diameter of a cartilage repairing implant. Because the defect sizing discs 211, 212, 213, 214 need to be placed over the cartilage surface of the damaged bone to measure the size of the damaged cartilage region, in a preferred embodiment, the multi-function device 200 is made of a transparent or substantially transparent material so that when the sizing discs are placed over the cartilage surface to determine the appropriate size that encompasses the damaged cartilage region, one can see through the sizing discs and see the cartilage surface behind the sizing discs. In some embodiments, at least the sizing disc portions of the multi-function device 200 are made of transparent or substantially transparent material.

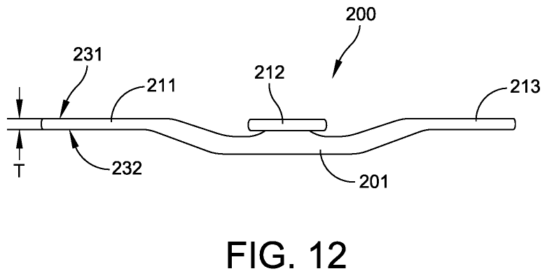

Referring to FIG. 12, each of the plurality of bone defect sizing discs 211, 212, 213, 214 has a first major surface 231 and a second major surface 232 that defines a thickness T that can be used for measuring or gauging the proudness of the cartilage repair implant. Unlike the sizing rings of the multi-function device 100 example, the sizing discs 211, 212, 213, 214 do not have an opening that can be placed over the protruding portion of the implant 500 to gauge the proudness, the sizing discs are placed next to the protruding portion of the implant 500 and visually or tactilely gauge the proudness against the thickness T of the sizing discs.

In some embodiments, each of the sizing discs 211, 212, 213, 214 on a given multi-function device can have different predefined diameters, for determining the appropriate size of cavity that needs to be formed in the bone to receive the cartilage repairing implant.

In some embodiments, one of the first and second major surfaces 231, 232 of the bone defect sizing discs can have a concave surface or a recessed surface for contacting a cartilage repair side on a bone. The sizing discs 211, 212, 213, 214 in the multi-function device 200 shown in FIG. 10 have recesses 221, 222, 223, 224, respectively. The sizing discs with recesses can be used in a similar manner to the sizing rings in the embodiment of FIG. 1 when the sizing discs are made of transparent material. As shown in FIG. 10, the sizing discs 211, 212, 213, 214 have ring-like ridges 211a, 212a, 213a, 214a along the perimeter of the recesses 221, 222, 223, 224, respectively, and they can be used in the similar manner as the sizing rings 111, 112, 113, 114 for sizing the cartilage repair site because one can see through the transparent material.

In the illustrated examples shown in FIGS. 10-18, the multi-function device comprises four bone defect sizing discs. In some other embodiments, however, the multi-function device 200 comprises two to six bone defect sizing discs. In some embodiments, the multi-function device 200 comprises three to five bone defect sizing discs.

In some embodiments, the bone defect sizing discs 211, 212, 213, 214 have a circular shape. In some embodiments, the bone defect sizing discs can have a non-circular shape such as an oval, a triangle, or a quadrilateral shape such as a square, a rectangle, a parallelogram, etc.

In some embodiments, each of the bone defect sizing discs 211, 212, 213, 214 comprise a hole 240 sized for receiving a guide pin or a guide wire. The hole 240 is located at the geometric center of the circular shaped sizing discs. After the proper size of a cavity to be drilled into the cartilage surface is determined using the sizing discs, while the selected sizing disc is being held over the damaged region of the cartilage, a guide pin (e.g. K-wire) can be placed through the hole 240 in the sizing disc and into the cartilage surface of the bone. This locates the guide pin in the center of the area on the cartilage where a cavity for the implant 500 should be drilled. The sizing disc is then removed from the site and a cavity is drilled into the cartilage surface of the bone using a cannulated drill bit with the aid of the placed guide pin.

Referring to FIG. 12, in some embodiments of the multi-function device 200, each sizing disc 211, 212, 213, 214 has a predetermined thickness T defined between the top first major surface 231 and the bottom second major surface of the sizing discs. The predetermined thickness T of the sizing disc can be used for assessing the proudness of the cartilage repairing implant 500 after the implant is implanted in the bone. The sizing disc can be placed next to the protruding portion of the implant 500 so that the proudness of the implant can be compared to the thickness T of the sizing disc. As mentioned above, depending on the need, the predetermined thickness T can represent a desired reference value for the proudness of the implant 500. In some embodiments, each of the sizing discs can be configured to have a different predetermined thickness for gauging varying implant proudness.

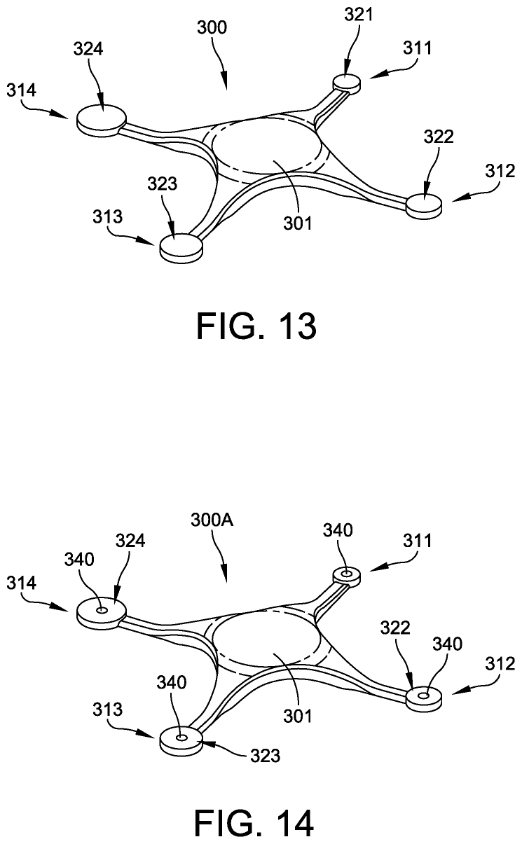

FIG. 13 shows a multi-function device 300 according to another embodiment. The multi-function device 300 comprises a body 301 and a plurality of bone defect sizing discs 311, 312, 313, 314, where each bone defect sizing disc has diameter of a predefined size that correlates to the diameter of a cartilage repairing implant. Because the defect sizing discs 311, 312, 313, 314 need to be placed over the cartilage surface of the damaged bone to measure the size of the damaged cartilage region, in a preferred embodiment, the multi-function device 300 is made of a transparent or substantially transparent material so that when the sizing discs are placed over the cartilage surface to determine the appropriate size that encompasses the damaged cartilage region, one can see through the sizing discs and see the cartilage surface behind the sizing discs. In some embodiments, at least the sizing disc portions of the multi-function device 300 are made of transparent or substantially transparent material.

In some embodiments, each of the sizing discs 311, 312, 313, 314 on a given multi-function device can have different predefined diameters, for determining the appropriate size of cavity that needs to be formed in the bone to receive the cartilage repairing implant.

In some embodiments, each of the sizing discs 311, 312, 313, 314 has a bone contacting surface 321, 322, 323, 324, respectively, that are concave surfaces to accommodate the general curvature of the cartilage surface on the bone.

In the illustrated example shown in FIG. 13, the multi-function device 300 comprises four bone defect sizing discs 311, 312, 313, 314. In some other embodiments, however, the multi-function device 300 comprises two to six bone defect sizing discs. In some embodiments, the multi-function device 300 comprises three to five bone defect sizing discs.

In some embodiments, the bone defect sizing discs 311, 312, 313, 314 have a circular shape. In some embodiments, the bone defect sizing discs can have a non-circular shape such as an oval, a triangle, or a quadrilateral shape such as a square, a rectangle, a parallelogram, etc. As with the other embodiments, the thickness of the bone defect sizing discs 311, 312, 313, 314 in the multi-function device 300 can have predefined thickness(es) to be used as a reference dimension to gauge the proudness of the cartilage repairing implant 500.

FIG. 14 is an illustration of a multi-function device 300A which is a variation of the multi-function device 300 of FIG. 13. The main difference is that in the device 300A, each of the bone defect sizing discs 311, 312, 313, 314 comprises a hole 340 sized for receiving a guide pin or a guide wire. The hole 340 is located at the geometric center of the circular shaped sizing discs 311, 312, 313, 314. As with the multi-function device 200, after the proper size of a cavity to be drilled into the cartilage surface is determined using the sizing discs 311, 312, 313, 314, while the selected sizing disc is being held over the damaged region of the cartilage, a guide pin (e.g. K-wire) can be placed through the hole 340 in the sizing disc and into the cartilage surface of the bone. This locates the guide pin in the center of the area on the cartilage where a cavity for the implant 500 should be drilled. The sizing disc is then removed from the site and a cavity is drilled into the cartilage surface of the bone using a cannulated drill bit with the aid of the placed guide pin. As with the other embodiments, the thickness of the bone defect sizing discs 311, 312, 313, 314 in the multi-function device 300A can have predefined thickness(es) to be used as a reference dimension to gauge the proudness of the cartilage repairing implant 500.

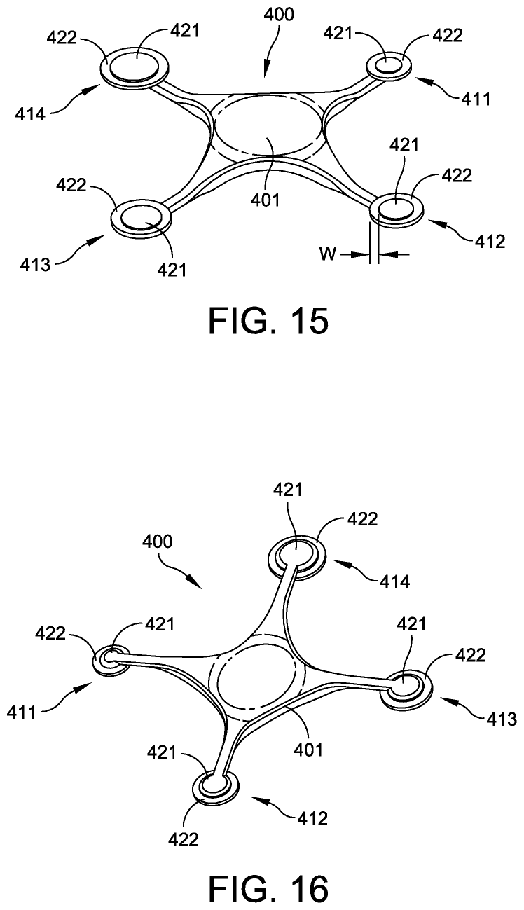

FIGS. 15-18 illustrate additional embodiments of the multi-function devices of the present disclosure. In FIG. 15 a multi-function device 400 is shown in perspective view from one of its two major sides. In FIG. 16, the multi-function device 400 is shown in perspective view from the other of its two major sides. The multi-function device 400 comprises a main body 401 and a plurality of bone defect sizing discs 411, 412, 413, 414. Each sizing disc comprises an inner region 421 and an outer region 422. Referring to the elevation view of the multi-function device 400 shown in FIG. 18, the inner region 421 is thicker than the outer region 422 and the inner region 421 has a predefined thickness T defined between the top surface 431 and the bottom surface 432. The predefined thickness T can be used for or assessing the proudness of the cartilage repairing implant 500 after the implant is implanted in the bone.

The predefined thickness T of the sizing disc can be about 2 mm. In some embodiments, each of the sizing discs 411, 412, 413, 414 on the multi-function device 400 has a different predefined thickness. This enables a single multi-function device to be used for measuring different proudness.

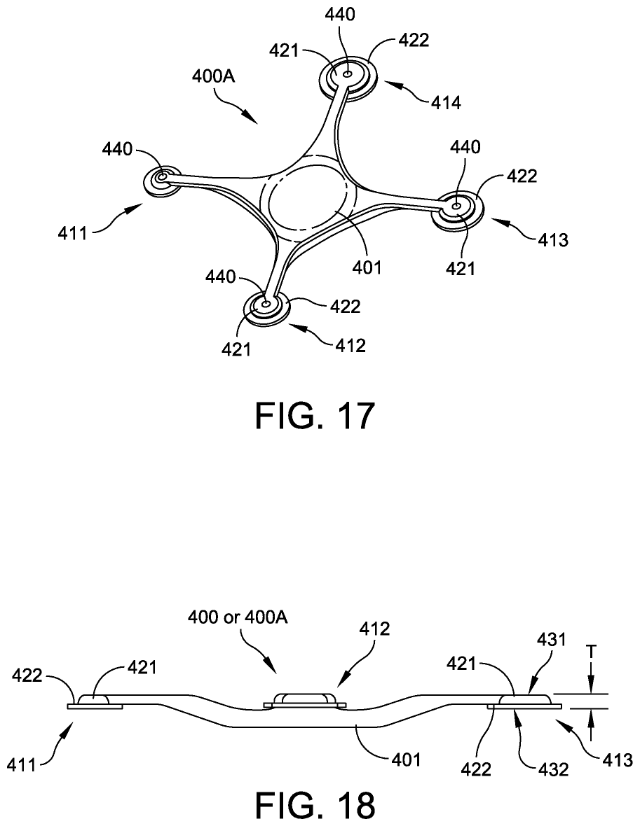

FIG. 17 shows a multi-function device 400A which is a variation of the multi-function device 400. In the multi-function device 400A, each of the sizing discs 411, 412, 413, 414 comprises a hole 440 sized for receiving a guide pin or a guide wire. The hole 440 is located at the geometric center of the circular shaped sizing discs 411, 412, 413, 414. As with the multi-function devices 200 and 300A, after the proper size of a cavity to be drilled into the cartilage surface is determined using the sizing discs 411, 412, 413, 414, while the selected sizing disc is being held over the damaged region of the cartilage, a guide pin (e.g. K-wire) can be placed through the hole 440 in the sizing disc and into the cartilage surface of the bone. This locates the guide pin in the center of the area on the cartilage where a cavity for the implant 500 should be drilled. The sizing disc is then removed from the site and a cavity is drilled into the cartilage surface of the bone using a cannulated drill bit with the aid of the placed guide pin. As with the other embodiments, the thickness of the bone defect sizing discs 411, 412, 413, 414 in the multi-function device 400A can have predefined thickness(es) to be used as a reference dimension to gauge the proudness of the cartilage repairing implant 500.

In both embodiments of the multi-function device 400 and 400A, the outer region 422 in one or more of the discs 411, 412, 413, 414 can have a predefined width W for evaluating the amount of bone material surrounding the intended location of the cartilage repairing implant. The width W is noted in FIG. 15.

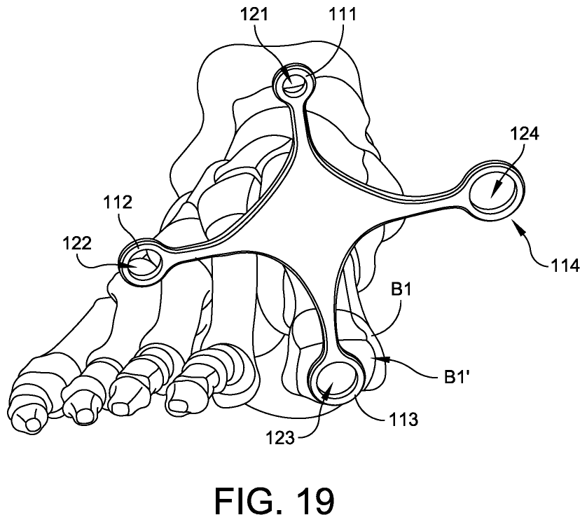

FIG. 19 is an illustration showing the example multi-function device 100 of FIG. 1 being used on a metatarsal bone B1. The bone defect sizing ring 113 is placed over the cartilage surface B1′ so that the opening 123 can be aligned over a damaged region (not identified on the Figure) of the cartilage surface B1′ to determine whether the opening 123 properly encompass the damaged region on the cartilage surface B1′.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

We claim:

1. A multi-function device comprising:
a plurality of bone defect sizing rings, wherein each bone defect sizing ring defines an opening of a predefined size that correlates to the perimeter shape and size of a cartilage repairing implant,
wherein the openings have different predefined sizes, for determining the appropriate size of the cartilage repairing implant for a cartilage repair site in a bone.

2. The device of claim 1, wherein the bone defect sizing rings and the corresponding opening have a circular shape, a substantially oval shape, a substantially quadrilateral shape, or a substantially triangular shape.

3. The device of claim 1, wherein at least the bone defect sizing rings are transparent.

4. The device of claim 1, wherein each loop has a top surface and a bottom surface and a predefined thickness between the top and bottom surfaces for assessing the proudness of the cartilage repairing implant after the implant is implanted in the bone.

5. The device of claim 4, wherein the predefined thickness is about 2 mm.

6. The device of claim 4, wherein each sizing ring has a different predefined thickness between the top and bottom surfaces.

7. The device of claim 1, wherein each sizing ring has a predefined width for assessing the amount of the bone material surrounding the intended location of the cartilage repairing implant in the bone.

8. The device of claim 7, wherein the predefined width is about 2 mm to 3 mm.

9. A multi-function device comprising:
a plurality of bone defect sizing discs, wherein each bone defect sizing disc has diameter of a predefined size that correlates to the diameter of a cartilage repairing implant,
wherein the discs have a first major surface and a second major surface, wherein, and
wherein the discs have different predefined diameters, for determining the appropriate size of the cartilage repairing implant for the cartilage repair site.

10. The device of claim 9, wherein one of the first and second major surfaces is concave or recessed for contacting a cartilage repair site on a bone.

11. The device of claim 9 comprises two to six bone defect sizing discs.

12. The device of claim 9, wherein the bone defect sizing discs have a circular shape, a substantially oval shape, a substantially quadrilateral shape, or a substantially triangular shape.

13. The device of claim 9, wherein at least the bone defect sizing rings are transparent.

14. The device of claim 9, wherein each of the bone defect sizing discs comprise a hole sized for receiving a guide pin or a guide wire.

15. The device of claim 9, wherein each disc has a predetermined thickness between the first major surface and the second major surface of the disc for assessing the proudness of the cartilage repairing implant after the implant is implanted in the bone.

16. The device of claim 9, wherein each disc has an inner region and an outer region, wherein the inner region is thicker than the outer region and has a predefined thickness for assessing the proudness of the cartilage repairing implant after the implant is implanted in the bone.

17. The device of claim 9, wherein each disc has a predefined thickness between the first major surface and the second major surface along the peripheral edge of the disc for assessing the proudness of the cartilage repairing implant after the implant is implanted in the bone.

18. The device of claim 16, wherein the predefined thickness is about 2 mm.

19. The device of claim 16, wherein each disc has a different predefined thickness between the top and bottom surfaces.

20. The device of claim 9, wherein each disc has an inner region and an outer region, wherein the outer region has a predefined width for evaluating the amount of bone material surrounding the intended location of the cartilage repairing implant.
